(19) United States
(12) Patent Application Publication
(10) Pub. No.: US 2014/0155698 A1
(43) Pub. Date: Jun. 5, 2014

(54) ENDOSCOPIC SURGICAL TOOL GUIDER PORT AND GAS EXHAUST VALVE FOR SURGICAL TOOL GUIDER PORT

(71) Applicant: O-Nam Seo, Gyeonggi-do (KR)

(72) Inventor: O-Nam Seo, Gyeonggi-do (KR)

(21) Appl. No.: 14/117,033

(22) PCT Filed: May 11, 2012
(86) PCT No.: PCT/KR12/03690

§ 371 Date: Nov. 12, 2013

(30) Foreign Application Priority Data
May 12, 2011 (KR) 10-2011-004308
May 12, 2011 (KR) 10-2011-0044307

(51) Int. Cl. A61B 17/50; A61B 17/34; A61B 10/04; A61B 17/02

(52) U.S. Cl. CPC A61B 17/50; A61B 17/0218; A61B 17/3423; A61B 10/04
USPC 600/204

(57) ABSTRACT

An endoscopic surgical tool guider port, installed in a surgical puncture site of a patient during an endoscopic surgery, wherein an extraction bag for containing and preserving extractions made during surgery protrudes from a tube body to the outside so that the surgery can be continued while the extractions made in the surgical process are directly contained and stored in the extraction bag, formed on the tube body, without requiring the opening or releasing of the guide port during the surgical process. A gas exhaust valve for a surgical tool guider port, and more particularly, a gas exhaust valve for a surgical tool guider port for promptly releasing toxic gases or smoke generated during the surgical process through a simple one-touch manipulation of pushing a button, thereby improving the convenience of use and enabling a smooth endoscopic surgery, compared to a conventional gas exhaust port used during surgery.

### CROSS REFERENCE TO RELATED APPLICATIONS AND CLAIM OF PRIORITY

This patent application is a National Phase application under 35 U.S.C. §371 of International Application No. PCT/KR2012/003690, filed May 11, 2012, which claims priority to Korean Patent Application Nos. 10-2011-0044307, filed May 12, 2011 and 10-2011-0044308, filed May 12, 2011, entire contents of which are incorporated herein by reference.

### BACKGROUND

1. Technical Field

The present invention relates to an endoscopic surgical tool guider port installed in a surgical puncture site to guide various surgical tools into an abdominal cavity, and more particularly to an endoscopic surgical tool guider port by which surgery can be continuously performed while extractions are temporarily preserved in an extraction bag formed on a tube body, without requiring the direct opening of the guider port several times to remove the extractions generated during surgery, enabling the endoscopic surgery to be performed more smoothly and promptly.

The present invention also relates to a gas exhaust valve for a surgical tool guider port, and more particularly, to a gas exhaust valve for a surgical tool guider port that is installed on an exhaust tube of the surgical tool guider port, used to guide various surgical tools into an abdominal cavity during endoscopic surgery, to easily release gas through a simple manipulation whenever needed during surgery, thereby enabling the endoscopic surgery to be performed more smoothly.

2. Description of the Related Art

In general, since a laparoscopic endoscopic surgery (also, referred to as ‘a minimum invasive operation’) requires a small surgical puncture site, compared to a traditional laparotomy, the resulting operation scar is cosmetically minimal and is treated along with the post-operative pain without complications. Furthermore, with rapid patient recovery speeds, hospital stays are short and patients can promptly return to their normal lifestyles. Thus, laparoscopic endoscopic surgeries have been frequently performed for nearly all diseases, except for some cancers.

Endoscopic surgery employs a method of piercing a small puncture site in the belly of a patient by using a surgical device called a trocar, in which at least one trocar is inserted into the belly and various surgical devices, such as forceps, cutting devices, internal organ extraction devices, and endoscopic cameras, are introduced into the belly through the trocar to perform various operations, such as gall bladder removals, gall bladder calculus removals, appendectomies, and general surgery.

In recent years, endoscopic surgeries with minimal scarring are being performed through the navel without trocars and puncture sites, as described above, resulting in faster patient recovery times. In general, endoscopic surgery through the navel is currently preferred because when the puncture site used for the operation is pierced via the naval of a human the resulting wound is minimally exposed and is oftentimes not visually recognizable as a scar after the wound heals.

A surgical tool guider port installed in a surgical puncture site, used to safely introduce various surgical tools into the abdominal cavity, is required for endoscopic surgery through the single surgical puncture site.

Endoscopic surgical tool guider ports having various configurations are currently disclosed. In particular, the applicant developed various endoscopic surgical tool guider ports that can be conveniently and safely installed in a surgical puncture site and the guider ports were patented. (Korean Patent Nos. 10-915882, 10-1027561, and 10-1027546)

However, extractions during surgical operations cannot be easily and efficiently processed by conventional surgical tool guider ports, including the patents of the applicant, and the surgical operation may be delayed or complicated.

For example, various extractions of internal organs, tumors, and gallstones are performed during endoscopic surgical processes. However, according to the related art, a separate extraction bag for organs, and the like, needs be introduced through an endoscopic surgical tool guider port, and this process can be troublesome with difficulties in securing a field of view, thereby causing the surgery to be cumbersome. Furthermore, since extractions are accomplished by opening an upper portion of an endoscopic surgical tool guider port (e.g., an open-top surgical tool guider port), or via releasing the endoscopic surgical tool guider port, the process becomes inconvenient and overall operation times are considerably prolonged.

Conventional surgical tool guider ports may have various configurations and basically include: a plurality of tool introduction ports for introducing various surgical tools at an upper end of the surgical tool guider port, a gas injection port for injecting gas (e.g., CO₂) to expand the abdomen of a patient, and a gas exhaust port for releasing toxic gases or smoke generated during surgery.

In particular, the gas exhaust port is necessary because when toxic gases or smoke are generated by burning flesh or blood vessels during the surgery, the toxic gases or smoke can be harmful to the patient if not promptly released. Furthermore, a smooth surgery can be compromised by poor visibility caused by the smoke or gases. Thus, a gas opening/closing valve in the gas exhaust port is generally provided to release toxic gases or smoke only when necessary.

However, gas opening/closing valves applied to conventional surgical tool guider ports have a structure used to rotate an operation lever, and it is troublesome to use the lever to open and close the valve. In particular, since toxic gases or smoke may be released several times during a surgical process, the inconvenience of use of the lever hinders an otherwise smooth surgery or delays surgery times.

### SUMMARY

The present invention has been made in an effort to solve at least one of the above-mentioned problems, and it is an object of the present invention to provide an endoscopic surgical tool guider port by which extractions made during surgery are contained in an extraction bag formed in a tube body to be conveniently processed so that the surgery can be performed more promptly and smoothly, without hindering the field of view or delaying operation times.

Another object of the present invention is to provide a gas exhaust valve for a surgical tool guider port by which toxic gases or smoke generated during a surgical process can be easily released through a simple one-touch manipulation so that an endoscopic surgery can be performed more smoothly.

In order to solve the above problems, according to one aspect of the present invention, there is provided an endoscopic surgical tool guider port installed in a surgical puncture site of a patient during an endoscopic surgery, used to safely guide various surgical tools introduced into a tube body through a tool introduction port provided at an upper portion thereof, into an abdominal cavity, wherein an extraction bag for containing and preserving the extractions made during the surgery is formed in the tube body and protrudes to the outside.

The extraction bag may be formed such that a lower end thereof is inclined downwardly and inward from an entrance of the extraction bag.

A plurality of independent storage chambers divided by partitions, for separating and preserving extractions, may be formed within the extraction bag.

According to another aspect of the present invention, there is provided a gas exhaust valve for a surgical tool guider port, installed in a surgical puncture site of a patient during an endoscopic surgery, to guide various surgical tools into an abdominal cavity, the gas exhaust valve including: a valve body fixedly installed in an exhaust tube of the surgical tool guider port and having a gas passageway; a packing member for closing or opening the gas passageway while being attached to or separated from the valve body; and an actuating member for separating the packing member from the valve body when an external force is applied, or for attaching the packing member to the valve body again when the external force is removed.

The actuating member may include: a rod member passing the valve body to protrude to opposite sides, the packing member being coupled to one of the protruding ends of the rod member; a button member formed at the other protruding end of the rod member, for moving the rod member forward when an external force is applied; and a resilient member installed within the valve body, for moving the rod member backwards when the external force is removed by providing a resilient restoring force to the rod member.

A body head attached to an end of the exhaust tube, when being inserted into and fixed to the exhaust tube, may protrude from an outer surface of one end of the valve body.

The packing member may be formed of a silicon material having a predetermined resiliency.

Use of the endoscopic surgical tool guider port of the present invention allows endoscopic surgeries to be performed more promptly and smoothly because extractions made during the surgery can be directly contained and stored in an extraction bag formed in a tube body, compared to having to use a separate extraction bag, which hinders the field of view, and having to open or release the guide port during the surgical process.

Furthermore, since various extractions can be separated and preserved, the utility of the guider port is improved and the guider port can be used more conveniently. Lastly, the guider port can be manufactured without increased manufacturing costs via a simple structure, thereby increasing economical efficiency.

With the gas exhaust valve for a surgical tool guider port of the present invention, toxic gas or smoke generated during surgical processes can be promptly released via a convenient one-touch pushing of a button member, resulting in improved convenience and smoother endoscopic surgeries, compared with the conventional gas exhaust port.

### BRIEF DESCRIPTION OF THE DRAWINGS

FIG. 1 is a perspective view showing an endoscopic surgical tool guider port according to one embodiment of the present invention;

FIG. 2 is a sectional view showing an extraction bag according to one embodiment of the present invention;

FIG. 3 is a sectional view showing an extraction bag according to another embodiment of the present invention;

FIG. 4 is a perspective view showing a gas exhaust valve according to one embodiment of the present invention;

FIG. 5 is a perspective view showing the gas exhaust valve when an external force is applied thereto according to one embodiment of the present invention;

FIG. 6 is a sectional view of the gas exhaust value of FIG. 4;

FIG. 7 is a sectional view of the resulting state of the gas exhaust valve of FIG. 5;

FIG. 8 is a view exemplifying an endoscopic surgical tool guider port, according to one embodiment of the present invention, in which the gas exhaust valve is installed; and

FIGS. 9 and 10 are views exemplifying the operation of the gas exhaust valve according to one embodiment of the present invention.

### DETAILED DESCRIPTION

Hereinafter, an endoscopic surgical tool guider port and a gas exhaust value for a surgical tool guider port according to exemplary embodiments of the present invention will be described in detail with reference to the accompanying drawings.

The embodiments of the present invention are provided to fully describe the present invention to those skilled in the art, and it should be noted that the shapes and sizes of the elements of the invention may be exaggerated in the drawings to clearly describe the elements.

In the following description of the embodiments, items with known functions and known configurations obvious to those skilled in the art will not be described when they may make the technical features of the present invention obscure.

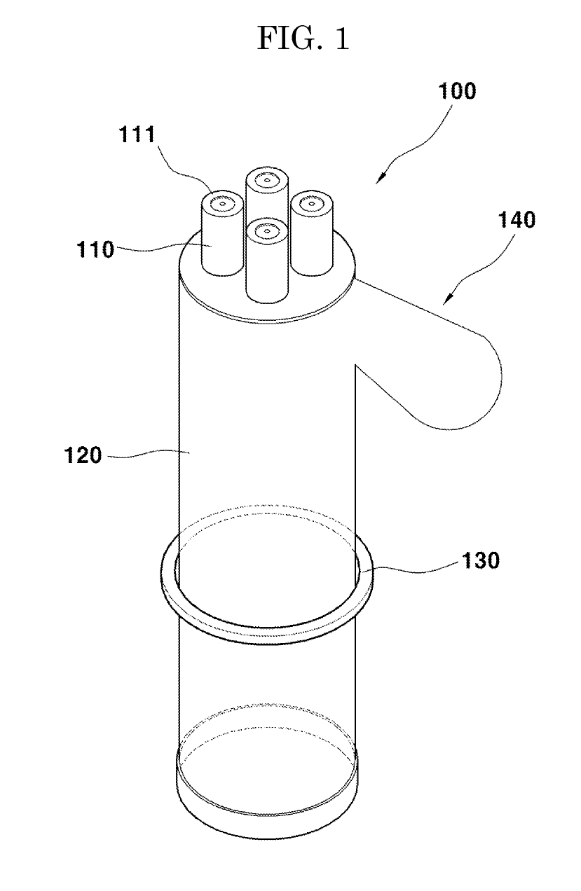

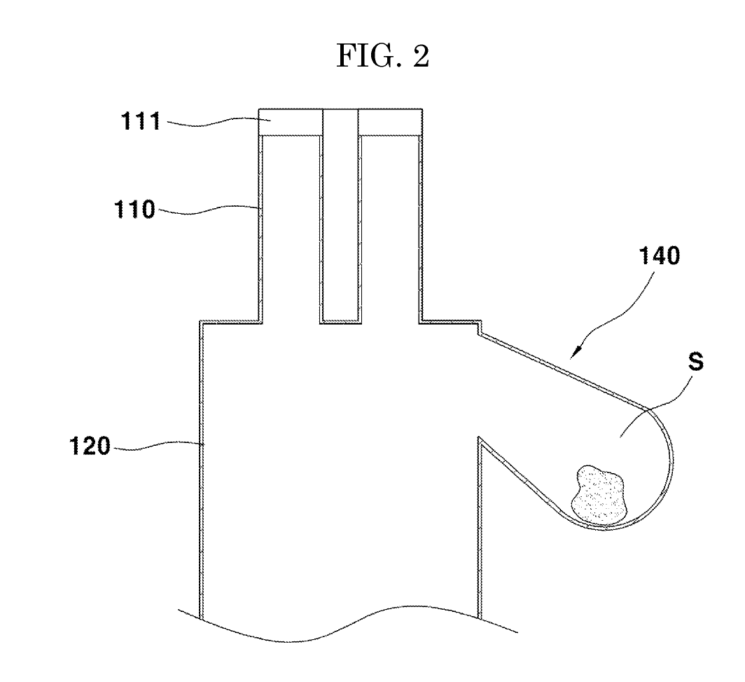

First, FIGS. 1 to 3 show the endoscopic surgical tool guider port 100 according to one embodiment of the present invention. Referring to the drawings, the endoscopic surgical tool guider port 100 (hereinafter, referred to as “guider port”), according to one embodiment of the present invention, may include a tool entry port 110, a tube body 120, a support ring 130 (FIG. 1), and an extraction bag 140 formed in the tube body 120.

The tool introduction port 110 is adapted to introduce various surgical tools, such as a nipper and an endoscope, into the tube body 120, and a valve unit 111 is used for easily introducing surgical tools, while maximally preventing the leakage of gas, and is provided at an upper end of the tool introduction port 110. The tube body 120 has a hollow cylindrical shape, and may be formed of a urethane sheet having excellent resiliency and durability.

The support ring 130 is an annular ring and is secured by the abdominal wall in the abdominal cavity after passing through the surgical puncture site of a patient. The support ring 130 supports the guider port 100 when the guider port 100 is installed. Thus, the support ring 130 may be formed of a resilient malleable material, such that the support ring 130 may easily pass through the surgical puncture site and may be subsequently be folded or shaped to be easily secured, while ultimately being restored to its original shape after passing back out of the surgical puncture site when the surgery in complete.

Various configurations of the tool introduction port 110, the tube body 120, and the support ring 130 are possible, and only one example is shown in the drawings. Thus, it will be understood that the tool introduction port 110, the tube body 120, and the support ring 130 may be manifested in various configurations and coupling relationships of surgical tool guider ports without being limited to the configurations shown in the drawings.

The extraction bag 140 is formed in the tube body 120 and has an interior space S for containing and preserving extractions made during surgery.

The extraction bag 140 protrudes from the tube body 120, so that the interior space of the tube body 120 is not affected even if the extractions are preserved in the extraction bag 140. Thus, the extraction bag 140 does not hinder the surgical field of view or the movement of surgical tools.

The extraction bag 140 may be integrated with the tube body 120 and thus may be formed of the same material as that of the tube body 120, more specifically, a urethane sheet having excellent malleability, resiliency, and durability.

As shown in the drawings, the extraction bag 140 may be formed such that the lower end thereof is hanging downward from an entrance portal (i.e., the part connected to the tube body 120). As such, with the lower end of the extraction bag hanging downward, the extractions are contained and preserved in the extraction bag 140 thereby preventing accidental withdrawal from the extraction bag 140 during surgical processes.

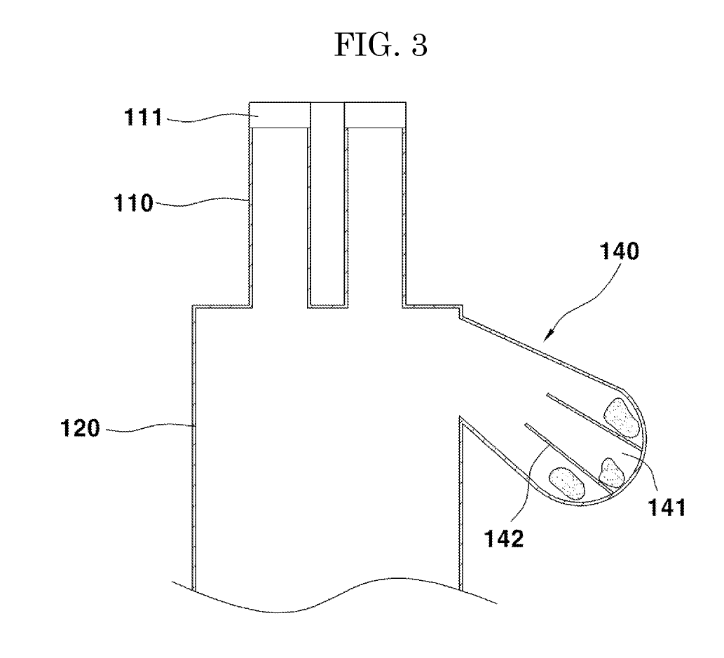

As shown in FIG. 3, a plurality of independent storage chambers 141 divided by partitions 142 may be formed in an interior portion of the extraction bag 140. As such, a plurality of extractions can be separated and preserved in the independent storage chambers 141 so as not to be mixed or contaminated by other extractions.

The configuration of the surgical tool guider port 100 according to one embodiment of the present invention has been described. While the surgical tool guider port 100 is installed in a surgical puncture site of a patient to perform endoscopic surgery, according to the present invention, various surgical tools can be introduced into the abdominal cavity within the tube body 120, through the tool introduction port 110.

If various extractions, such as tumors and gallstones, are made during an endoscopic surgery, the extractions can directly be contained in the extraction bag 140 formed in the tube body 120 thereby streamlining the surgery, instead of introducing a separate extraction bag to contain and preserve the extractions, or opening or releasing the surgical tool guider port 100 to remove the extractions, and then reinstalling the surgical tool guider port 100 to resume the surgery.

When the extractions need to be separated and preserved, an operation can be performed while the extractions are conveniently separated and preserved in the independent storage chambers 141 of the extraction bag 140.

It can be seen that if an endoscopic surgery is performed by using the surgical tool guider port 40 according to the present invention, extractions are preserved very conveniently so that the endoscopic surgery can be performed more smoothly without causing a delay in an operation time due to processing of the extractions at all.

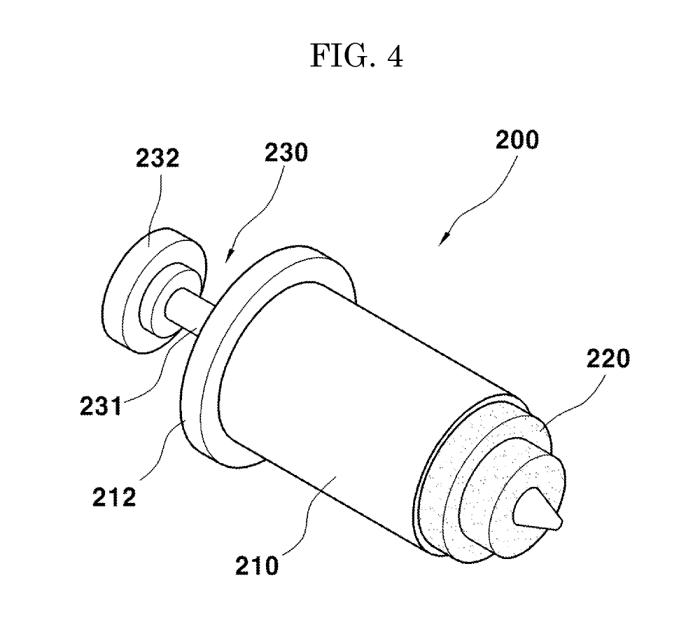

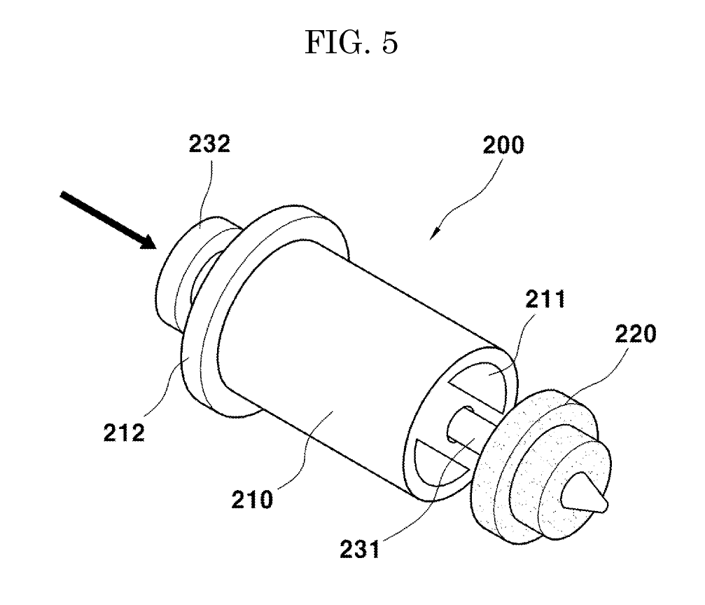

FIGS. 4 to 7 show the gas exhaust valve 200 according to one embodiment of the present invention. As shown, the gas exhaust valve 200 may include a valve body 210, a packing member 220, and an actuating member 230.

The valve body 210 has a substantially cylindrical shape. A gas passageway 211 is formed within the valve body 210 such that gas passes through the gas passageway 211, and a body head 212 protrudes from an outer surface of one end of the valve body 210.

The packing member 220 is provided at one end of the valve body 210 to selectively open and close the gas passageway 211 of the valve body 210. More specifically, the packing member 220 blocks and closes the gas passageway 211 when attached to one end of the valve body 210, and opens the gas passageway 211 when separated from one end of the valve body 210.

The packing member 220 may be formed of a silicon material having a predetermined resiliency to allow proper sealing when the packing member 220 is attached to the valve body 210 and closes the gas passageway 211.

When an external force is applied, the actuating member 230 separates the packing member 220 from the valve body 210 and opens the gas passageway 211, and when the external force is removed, the packing member 220 is attached to the valve body 210 and closes the gas passageway 211.

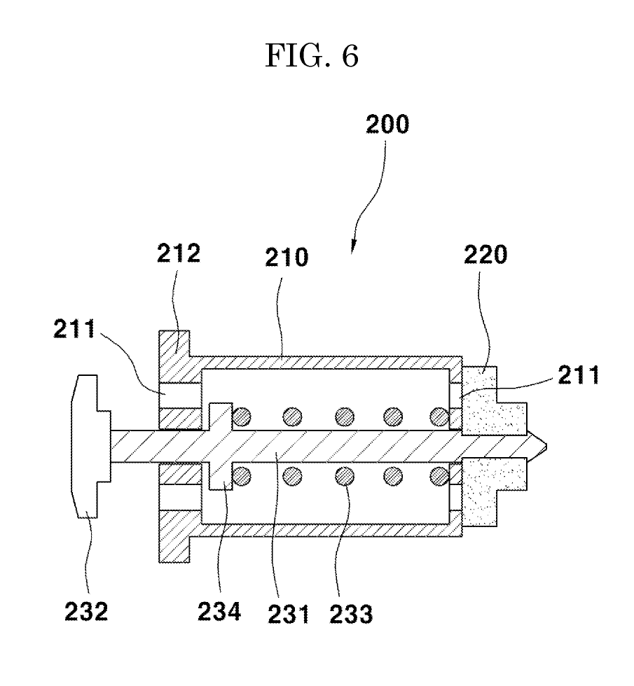

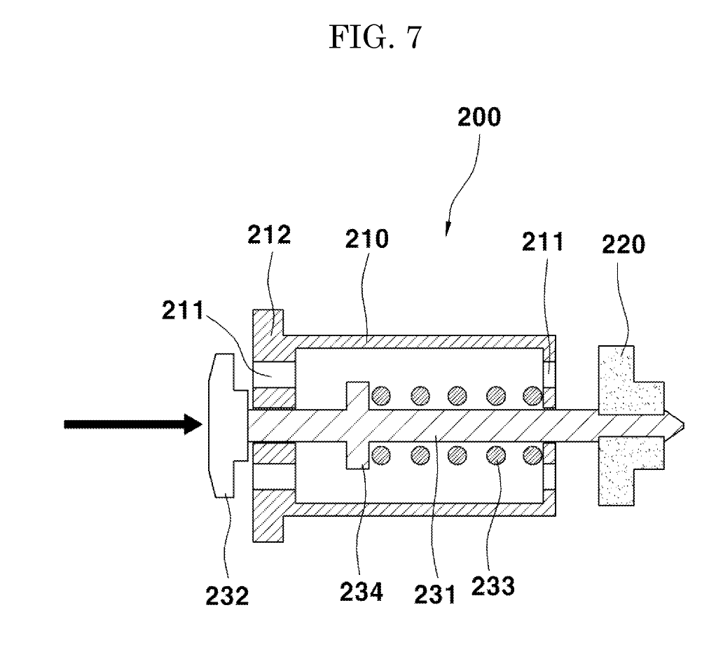

The actuating member 230 may include a rod member 231, a button member 232, and a resilient member 233 (FIGS. 6 and 7).

The rod member 231 passes through the valve body 210 to protrude from opposite sides of the valve body 210, and the packing member 220 is fixedly coupled to one of the protruding ends of the rod member 231. With the packing member 220 being fixedly coupled to the rod member 231, the packing member 220 moves in conjunction with the rod member 231.

The button member 232 is formed on the opposite end of the rod member 231 (i.e., opposite to the protruding end of the rod member 231 with packing member 220) and the button member 232 is where an external force can be applied by the user, so that the connected rod member 231 moves forward when the external force is applied.

The resilient member 233 (FIG. 6) is installed within the valve body 210 such that the rod member 231 is moved backwards to its original position when the external force applied to the button member 232 is removed.

The resilient member 233 should provide a resilient restoring force to the rod member 231, and the resilient member 233 may be manifested in a spring that is fitted with the rod member 231 while being supported by a support 234 formed on the rod member 231.

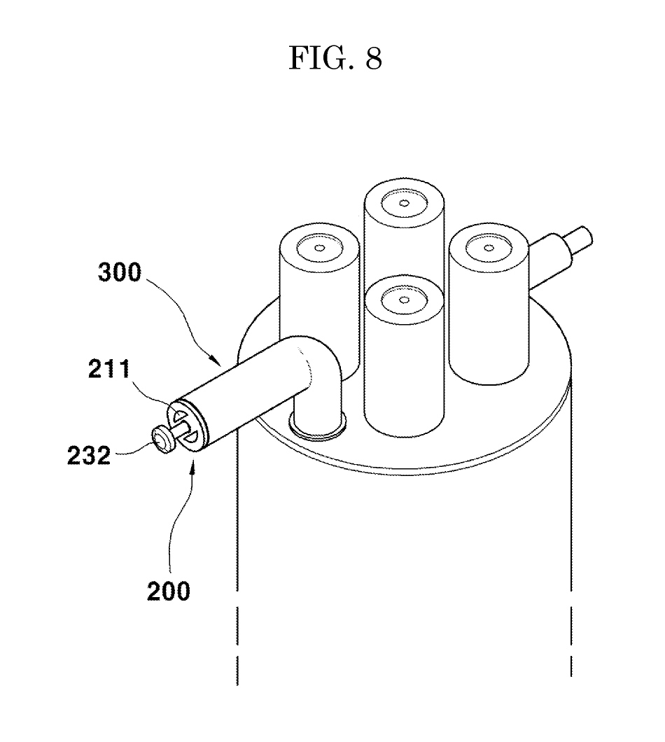

The gas exhaust valve 200, according to one embodiment of the present invention, is fixedly installed in an exhaust tube 300 (FIG. 8) provided at an upper end of the surgical tool guider port 100. A detailed description thereof will be omitted for clarity. It is to be noted that the installation of the gas exhaust valve 200, according to the present invention, is without limitation with respect to configuration on the surgical tool guider port 100.

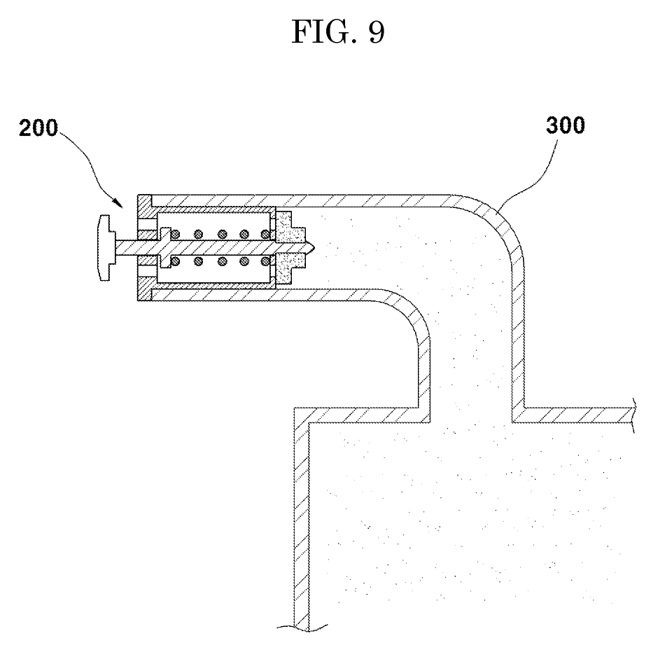

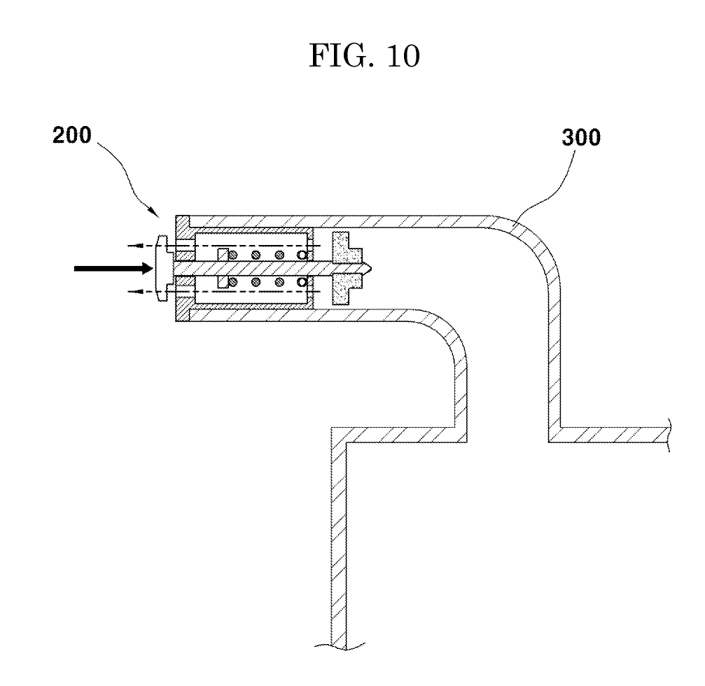

FIG. 8 exemplifies a state in which the gas exhaust valve 200, according to the present invention, is installed in the exhaust tube 300 of the surgical tool guider port 100, and FIGS. 9 and 10 exemplify the operation of the gas exhaust valve 200. Hereinafter, the installation and operation of the gas exhaust valve 200 will be described with reference to the drawings.

First, the gas exhaust valve 200 of the present invention is installed into the exhaust tube 300, wherein the valve body 210 is inserted into the exhaust tube 300. The protruding body head 212 of the valve body 210 can be fixed to the end of the exhaust tube 300 firmly.

If the exhaust tube 300 is fixedly installed, the gas exhaust valve 200 is normally in a state in which the packing member 220 is attached to the valve body 210 and the gas passageway 211 is closed to stop the leakage of gas.

When the button member 232 is pushed, the rod member 231 is moved forward, as shown in FIG. 10, resulting in the packing member 220, coupled to the rod member 231, being moved in conjunction with the rod member 231 and being separated from the valve body 210 at the same time. Thus, the gas passageway 211 of the valve body 210 is opened, and toxic gases or smoke in the opened gas passageway 211 are promptly released.

When the release of gases and smoke is completed, the applied external force is removed by releasing button member 232.

Then the rod member 231 is moved backwards while the compressed resilient member 233 provides a resilient restoring force to the rod member 231, and the packing member 220 closes the gas passageway 211 resulting in attachment to the valve body 210 and closing of the system.

The gas exhaust valve 200, according to the present invention, can conveniently release or contain gas through the simple manipulation of a one-touch button, and contributes to a smoother endoscopic surgery through the improved convenience of use.

Although the embodiments of the present invention have been described, the technical scope of the present invention is not limited by the embodiments and the contents of the drawings. Modified equivalents by those skilled in the art to which the present invention pertains fall within the scope of the present invention.

1. An endoscopic surgical tool guider port installed in a surgical puncture site of a patient during an endoscopic surgery to safely guide various surgical tools introduced into a tube body through a tool introduction port provided at an upper portion into an abdominal cavity, the endoscopic surgical tool guider port comprising:
an extraction bag formed in the tube body for containing and preserving extractions made during surgery, wherein the extraction bag protrudes to an outside.

2. The endoscopic surgical tool guider port of claim 1, wherein the extraction bag has a lower end hanging downward from an entrance of the extraction bag.

3. The endoscopic surgical tool guider port of claim 1, wherein a plurality of independent storage chambers divided by partitions for separating and preserving extractions are formed within the extraction bag.

4. A gas exhaust valve for a surgical tool guider port installed in a surgical puncture site of a patient during an endoscopic surgery to guide various surgical tools into an abdominal cavity, the gas exhaust valve comprising:
a valve body fixedly installed in an exhaust tube of the surgical tool guider port and having a gas passageway;
a packing member for closing or opening the gas passageway while being attached to or separated from the valve body; and
an actuating member for separating the packing member from the valve body when an external force is applied thereto, or for attaching the packing member to the valve body again when the external force is removed.

5. The gas exhaust valve of claim 4, wherein the actuating member comprises:
a rod member bi-directionally protruding by passing through the valve body, the packing member being coupled to one of protruding ends of the rod member;
a button member formed at the remaining end of the protruding ends of the rod member for moving the rod member forwards when an external force is applied thereto; and
a resilient member installed within the valve body for moving the rod member backwards when the external force is removed by providing a resilient restoring force to the rod member.

6. The gas exhaust valve of claim 4, wherein a body head attached to an end of the exhaust tube when being inserted into and fixed to the exhaust tube protrudes from an outer surface of one end of the valve body.

7. The gas exhaust valve of claim 4, wherein the packing member is formed of a silicon material having a predetermined resiliency.

8. The endoscopic surgical tool guider port of claim 2, wherein a plurality of independent storage chambers divided by partitions for separating and preserving extractions are formed within the extraction bag.

9. The gas exhaust valve of claim 5, wherein the packing member is formed of a silicon material having a predetermined resiliency.

10. The gas exhaust valve of claim 6, wherein the packing member is formed of a silicon material having a predetermined resiliency.
